(19) United States
(12) Patent Application Publication
(10) Pub. No.: US 2024/0417668 A1
(43) Pub. Date: Dec. 19, 2024

(54) BIOREACTOR SYSTEM FOR PRODUCTION OF CELL-CULTURED MEAT AND RELATED METHOD

(71) Applicant: The Trustees of Boston College, Chestnut Hill, MA (US)

(72) Inventors: Bryan RANGER, Somerville, MA (US); Glenn R. GAUDETTE, Holden, MA (US); Richard THYDEN, Sutton, MA (US); Luke R. PERREAULT, Blackstone, MA (US); Antonio C. FREITAS DOS SANTOS, Waltham, MA (US)

(21) Appl. No.: 18/742,957

(22) Filed: Jun. 13, 2024

Related U.S. Application Data
(60) Provisional application No. 63/508,218, filed on Jun. 14, 2023.

(51) Int. Cl. C12M 1/34; C12M 1/12; C12M 1/36; C12N 5/04

(52) U.S. Cl. CPC C12M 41/36; C12M 25/14; C12M 41/48; C12N 5/04

(57) ABSTRACT

A bioreactor includes a chamber for containing a reaction, which can be a decellularization reaction, a proliferation reaction, or a differentiation reaction. An ultrasound probe is configured to emit ultrasonic pulses into the chamber to detect the degree of decellularization, proliferation, or differentiation. The ultrasound probe may emit pulses with a frequency of up to about 100 MHz. Additionally, the ultrasound probe can be connected to a processor with memory and software to provide digital images indicative of the reaction's progress. The software can analyze these images to detect the reaction's degree and may include a trained machine learning software module.

### CROSS-REFERENCE TO RELATED APPLICATIONS

This application claims the benefit of priority to U.S. Provisional Application Ser. No. 63/508,218, filed Jun. 14, 2023, and which is incorporated by reference in its entirety.

### BACKGROUND

### Field

The present disclosure relates generally to the production of cell-cultured meat, and, particularly, to the use of acoustic pulses as a means of real-time, non-destructive quality control in the production of cell-cultured meat.

### Description of the Problem and Related Art

The animal agriculture industry has long been a primary producer of food for Americans and many other countries. However, food production by means of conventional agriculture accounts for one of the highest contributors to environmental impact and resource usage both domestically and globally. As an example, to produce a quarter pound of beef requires 74.5 ft² of land and 52.8 gallons of water. In addition, cattle are responsible for 23% of the methane production in the US and 9.4% of anthropogenic heat-trapping gasses in the world. The production of meat, eggs and dairy products generates vast quantities of wasted energy and is being met with increasing societal concerns over biosecurity (particularly the risk of zoonotic pandemics), animal waste, human pathogens, treatment of animals, and labor conditions for industry workers. The current system as it stands is not sustainable, especially with an increasing and ever more affluent global population. American agriculture and manufacturing is also at risk of losing its competitive edge in global markets, as China and Chinese AI firms are making major investments in 4th Industrial Revolution technologies that threaten US geopolitical leadership and competitiveness across a host of industries.

Cellular agriculture, the biomanufacturing of animal meat products, presents a novel and promising alternative to current meat production by conventional agriculture. Cellular agriculture can alleviate social and environmental challenges by minimizing the volume of animals in the production process, shorten the supply chain and dramatically changing the pattern of human interactions with nutrient sources. This bottom-up approach utilizes cells to manufacture the same biophysical products conventionally produced by whole organisms (e.g., animals). Other than the benefits of reducing feedstock, land and water use, the cellular agriculture approach offers a number of other significant consumer benefits, including ‘cleaner’ foods grown in controlled environments without antibiotics, pesticides and other contaminants, precise nutritive value through engineering content of tailorable foods, and the potential for local sourcing to reduce transportation costs and support regional food economies.

All animal agriculture is sustained through plant material and energy, but at a deeply inefficient rate of conversion from pre-harvest biomass to final consumer products. Efforts have accordingly begun to exploit the plant kingdom properties to accomplish specific tasks around animal cell culture. In addition to thinking about plants as food, plants are being developed as edible scaffolds for animal cell growth and assembly. The similarities and apparent biocompatibility of plant ECM (extracellular matrix) have shown that plants and their innate vasculatures could serve as perfusable scaffolds for engineering animal tissue. Decellularization techniques are used to remove the plant cells from multiple plant types, all with different hierarchical geometries. The vasculature remained patent after decellularization, as demonstrated by dye, microsphere and blood perfusion. The decellularized plant scaffolding also supported bovine myocyte precursor cell growth and differentiation. As plant scaffolds can be manufactured in virtually unlimited supply from a range of plants, this provides a low-cost, edible scaffolds for growing and assembling muscle tissue from different types of meat, poultry and fish.

Conventional decellularization methods use solutions that are not considered safe for use in food, such as organic solvents (hexanes) and detergents (triton X-100 (TX100)). These are destructive and would not be feasible for a scaled-up QC process for bioreactors. Polysorbate-20 (PS20) is a nonionic detergent that is used in various food processing steps as a wetting agent, emulsifier, and flavoring agent and is a REG substance according to the FDA.

Ultrasound imaging is commonly used in a wide variety of fields to visualize, test, and measure different parameters that are often invisible or hard to determine with the naked eye. In particular, it stands out as a non-intrusive and non-destructive manner to analyze and characterize a plethora of substances. Some of the other benefits of using ultrasound imaging as opposed to other imaging modalities are how quickly useful images can be generated, how relatively cheap the imaging is, and yet how relatively precise the results are. Ultrasound imaging has been shown to be viable for bioprocess monitoring and control, including for chemical analysis.

Ultrasound imaging has utility in monitoring inorganic materials without making contact with them. For example, calculations for the structural integrity of stainless-steel plates in high-temperature atmospheres are important to manage to avoid impending damages. In addition, ultrasound is very practical for monitoring various aspects of biological materials without damaging or altering them. For example, in biomedical applications such as imaging trabecular and cortical bones, ultrasound has potential to diagnose bone fragility by predicting bone pathology by looking at the attenuation of ultrasonic waves as they propagate through the bone, monitor fatigue microdamage via nonlinear resonant ultrasound spectroscopy (NRUS), or more broadly assess material properties and detect defects with quantitative laser ultrasound visualization (QLUV). It is essential that these processes be non-invasive and non-destructive, so as not to damage the bone, which is why these various ultrasound techniques are so useful.

In addition, tissue engineering and biomaterials is a pertinent topic with regards to ultrasound monitoring, since traditional methods are destructive, time-and-cost inefficient, and do not allow time-lapse measurements, making ultrasound a prime candidate for non-destructive characterizations of such tissue engineered projects. Furthermore, high-frequency quantitative ultrasound techniques can enable volumetric characterization of the structural, biological, and mechanical properties of engineered tissues during fabrication and post-implantation.

### SUMMARY

For purposes of summary, certain aspects, advantages, and novel features are described herein. It is to be understood that not necessarily all such advantages may be achieved in accordance with any one particular embodiment. Thus, the apparatuses or methods claimed may be embodied or carried out in a manner that achieves or optimizes one advantage or group of advantages as taught herein without necessarily achieving other advantages as may be taught or suggested herein.

In accordance with embodiments, a bioreactor is provided comprising a chamber for containing a reaction, which can be a decellularization reaction, a proliferation reaction, or a differentiation reaction. The bioreactor includes an ultrasound probe configured to emit ultrasonic pulses into the chamber to detect a degree of decellularization, proliferation, or differentiation. In some embodiments, the ultrasound probe emits pulses with a frequency of up to about 100 MHz. Additionally, the ultrasound probe may be connected to a processor with memory and software operative to provide digital images indicative of the reaction's degree. The software may also be capable of analyzing the digital images to detect the degree of decellularization, proliferation, or differentiation, and may include a trained machine learning software module.

A method of manufacturing cell-cultured meat includes decellularizing tissue and using ultrasonic pulses to detect the degree of decellularization. Primer cells are seeded onto a scaffold, and ultrasonic pulses are again used to detect cellular confluence. Following this, cells populating confluent scaffolds are induced to differentiate, with the degree of differentiation detected by propagating ultrasonic pulses. The method further involves acquiring ultrasound image data, defining intensity thresholds, and segmenting data based on these thresholds, which can be performed by trained machine learning software. Additionally, the method can be applied to plant tissues such as spinach, broccoli, and aloe, and executed in a real-time, continuous manner for a production line with quality control monitoring.

A system for manufacturing cell-cultured meat includes a decellularization bioreactor, an ultrasound probe to detect the degree of decellularization, a proliferation bioreactor with a scaffold for primer cells, and an ultrasound probe to monitor confluence. Additionally, a differentiation bioreactor with an ultrasound probe assesses cell differentiation. The ultrasound probe can emit pulses up to about 100 MHz. The system can handle plant tissues such as spinach, broccoli, and aloe. It also features a computer-based system with program instructions for processing ultrasound image data, defining thresholds based on intensity, and segmenting data, which can be enhanced by machine learning software modules.

### BRIEF DESCRIPTION OF THE DRAWINGS

The apparatus and method are described with reference to the accompanying drawings. In the drawings, like reference numbers indicate identical or functionally similar elements. Additionally, the left-most digit(s) of a reference number identifies the drawing in which the reference number first appears.

FIG. 1 depicts an exemplary bioreactor for containing a decellularization reaction, a proliferation reaction, or a differentiation reaction.

FIG. 2 illustrates an exemplary system for production of cell-cultured meat.

FIG. 3 is a flow chart of an exemplary method for the production of cell-cultured meat.

FIG. 4 is a flow chart of an exemplary method for detecting the degree of decellularization, or proliferation, or differentiation.

FIG. 5 is a functional block diagram of an exemplary computer system.

FIG. 6 is a functional block diagram of software programs that may be configured on the memory of computer system of FIG. 5.

### DETAILED DESCRIPTION

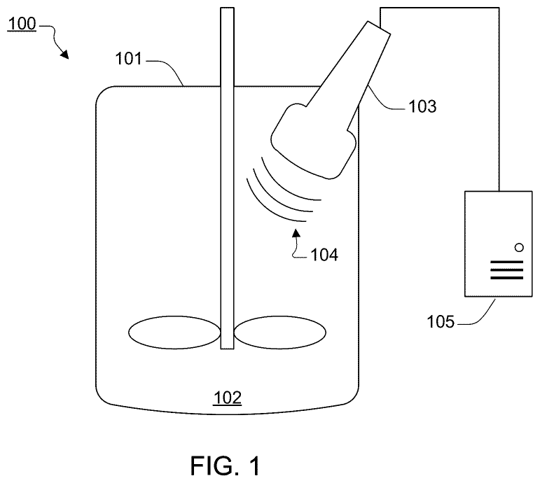

The various embodiments of the bioreactor system and method, and their advantages, are best understood by referring to FIGS. 1 through 6 of the drawings. The elements of the drawings are not necessarily to scale, emphasis instead being placed upon clearly illustrating the novel features and principles of operation. Throughout the drawings, like numerals are used for like and corresponding parts of the various drawings.

Furthermore, reference in the specification to “an embodiment,” “one embodiment,” “various embodiments,” or any variant thereof means that a particular feature or aspect described in conjunction with the particular embodiment is included in at least one embodiment. Thus, the appearance of the phrases “in one embodiment,” “in another embodiment,” or variations thereof in various places throughout the specification are not necessarily all referring to its respective embodiment.

With reference to FIG. 1, an exemplary bioreactor 100 is designed to facilitate various biological reactions in the production of cell-cultured meat, specifically, decellularization, proliferation, and differentiation reactions. The bioreactor 100 comprises a vessel 101 that defines a chamber 102 suitable for containing these reactions. An ultrasound probe 103 is associated with bioreactor 100, and is configured to emit ultrasonic pulses 104 into the chamber 102. These pulses 104 are used for detecting the degree of decellularization, proliferation, or differentiation occurring within the chamber 102. This non-invasive method allows for real-time monitoring of the reaction's progress.

The ultrasound probe 103 is designed to emit pulses 104 at frequencies reaching up to about 100 MHZ. Additionally, the ultrasound probe 103 has a detection range of up to about 7 cm, ensuring that it can effectively monitor reactions within a substantial volume of the chamber 102. These features are for providing accurate and comprehensive monitoring of the biological processes within the bioreactor.

Data collected by the ultrasound probe 103 is provided to a computer-based system 105 equipped with a processor and a memory configured with specialized software, described in detail hereafter. Probe 103 is in communication with the computer system 105, either wirelessly (e.g., WiFi, Bluetooth, etc.) or wired. This software is capable of generating one or more digital images that are indicative of the degree of decellularization, proliferation, or differentiation. In a preferred embodiment, the computer system 105 utilizes trained machine learning algorithm trained to accurately analyze and assess the degree to which the appropriate reaction is taking place. This feature allows for precise, automated monitoring of the reactions and promotes a continuous process for the production of cell-cultured meat.

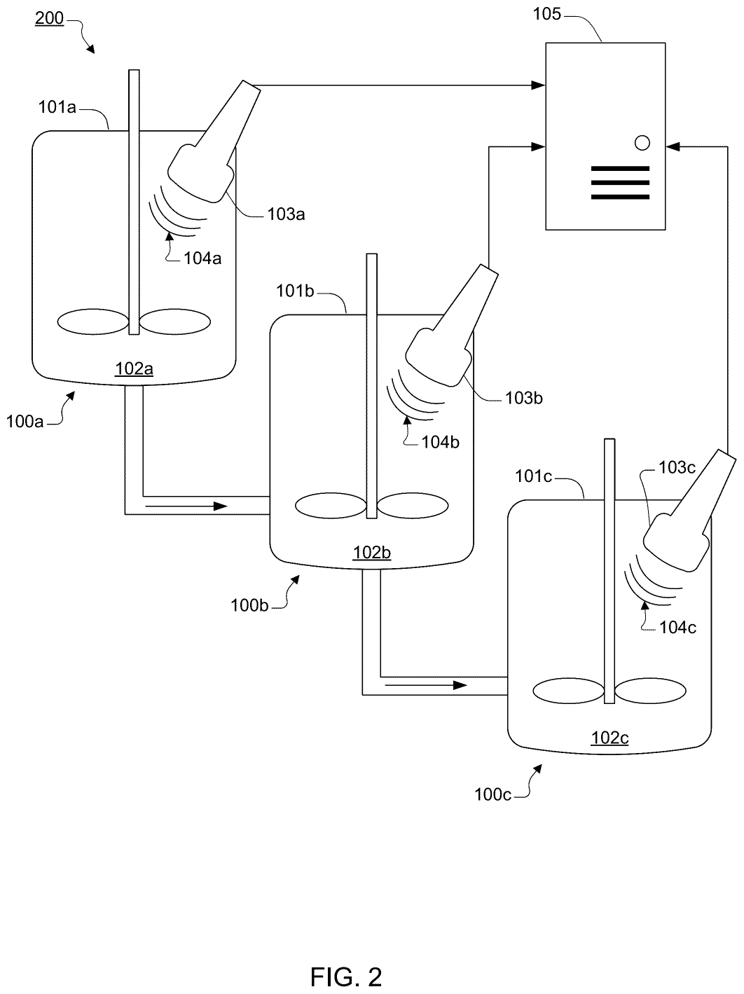

A bioreactor system 200, illustrated in FIG. 2, is designed for the production of cell-cultured meat, comprising a series of bioreactors 100a-c for containing and facilitating various types of reactions in cell-cultured meat production, such as decellularization, proliferation, and differentiation. Each bioreactor 100a-c is associated with an ultrasound probe 103a-c.

The decellularization bioreactor 100a is configured to contain and facilitate the decellularization of tissue and comprises a decellularization reaction vessel 101a and defines a decellularization reaction chamber 102a. The decellularization bioreactor 100a is associated with an ultrasound probe 103a adapted to propagate ultrasonic pulses 104a into the chamber 102a at the reaction therein. The tissue may be any suitable tissue. In one embodiment, tissue is a plant tissue. In a preferred embodiment, the plant tissue is aloe, spinach, or broccoli.

The proliferation bioreactor 100b is configured to contain a scaffold obtained from the decellularization reaction and seeded with primer cells to support cell growth and comprises a proliferation reaction vessel 101b that defines a proliferation reaction chamber 102b. The proliferation bioreactor 100b is associated with an ultrasound probe 103b adapted to propagate ultrasonic pulses 104b into the proliferation chamber 102b at the reaction therein.

The differentiation bioreactor 100c contains and facilitates a reaction for differentiation of cells populating a confluent scaffold obtained from the proliferation reaction. The differentiation bioreactor 100c comprises a differentiation vessel 101c that defines a reaction chamber 102c. The differentiation bioreactor 100c is associated with an ultrasound probe 103c adapted to propagate ultrasonic pulses 104c into the differentiation reaction chamber 102c at the reaction therein.

The ultrasound probes 103a-c are configured to emit pulses with a frequency of up to about 100 MHz and each has a detection range, based upon frequency and reaction chamber size, suitable to measure the depth of the reaction as would be appreciated by those skilled in the relevant arts. Additionally, each ultrasound probe 103a-c is responsive to a computer system 105. Each probe 103a-c is in communication with the computer system 105, either wirelessly (e.g., WiFi, Bluetooth, etc.) or wired. Computer system 105 includes a processor, and a memory configured with software operative to generate digital images indicative of the reaction stages. In a preferred embodiment, the computer system 105 is configured with a machine learning module trained to detect the degrees of reaction.

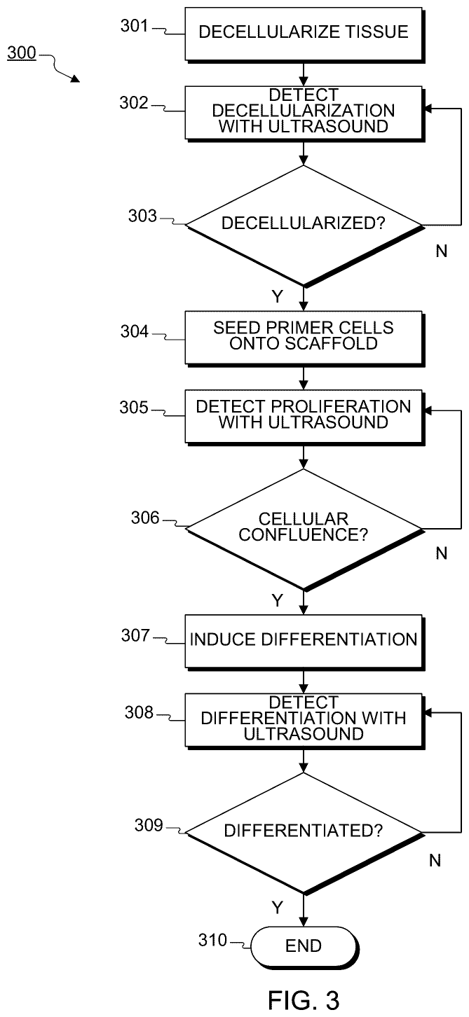

It will be appreciated, this system 200 facilitates the efficient and controlled production of cell-cultured meat with the aid of ultrasound technology and, preferably, machine learning algorithms. The method for manufacturing cell-cultured meat 300, illustrated with a flow chart in FIG. 3, involves several steps, starting with decellularizing tissue 301 in the decellularization bioreactor 100a. Decellularization may be performed by exposing candidate tissue to decellularization agents including Sodium Dodecyl Sulfate, Polysorbate 20, and Bleach, for up to 1 week.

At step 302, ultrasonic pulses 104a are propagated into chamber 102a into the decellularization chamber 102a via the probe 103a to detect the degree of decellularization. If decellularization has occurred to a sufficient degree 303, the resulting decellularized scaffold is transferred to the proliferation bioreactor 100b. If not, the decellularization reaction is allowed to proceed.

After decellularization is complete, at step 304, skeletal muscle progenitor cells are seeded onto the resulting decellularized scaffold and a proliferation reaction proceeds in the proliferation bioreactor 100b. Step 305 has the ultrasound probe 103b propagating ultrasonic pulses 104b into the proliferation chamber 102b to detect a degree of cellular confluence. If cellular confluence has occurred to a sufficient degree 306, the resulting cell structure is transferred to the differentiation bioreactor 100c. If not, the proliferation reaction is allowed to proceed.

In the differentiation chamber 102c, cells populating confluent scaffolds are induced to differentiate into muscle cells, and fat cells at Step 307. The degree of differentiation is detected, at Step 309, by ultrasound probe 103c propagating ultrasonic pulses 104c into the differentiation chamber 102c. If differentiation has occurred to a sufficient degree 309, the process ends 310. If not, the differentiation reaction is allowed to proceed.

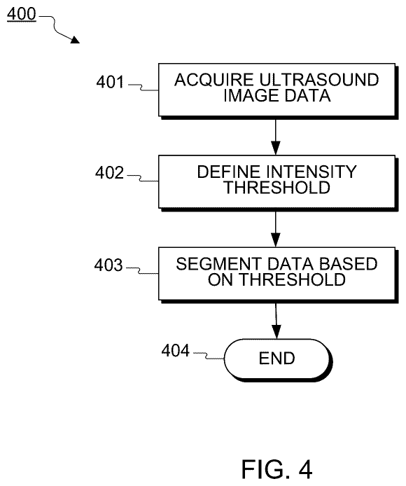

Turning to FIG. 4, a method of detecting decellularization, proliferation or differentiation 400 is depicted by another flow chart where, at Step 401, two-dimensional ultrasound image data is acquired by the computer system 105. The system 105 is configured to define a threshold based on intensity at Step 402, and a portion of the data is segmented based on the threshold (403). This segmentation can be performed by a trained machine learning module. The method can be executed as a real-time and continuous method, capable of providing a continuous production line for cell-cultured meat with real-time quality control monitoring, including machine learning of the degree of differentiation for longer than one week of continuous production.

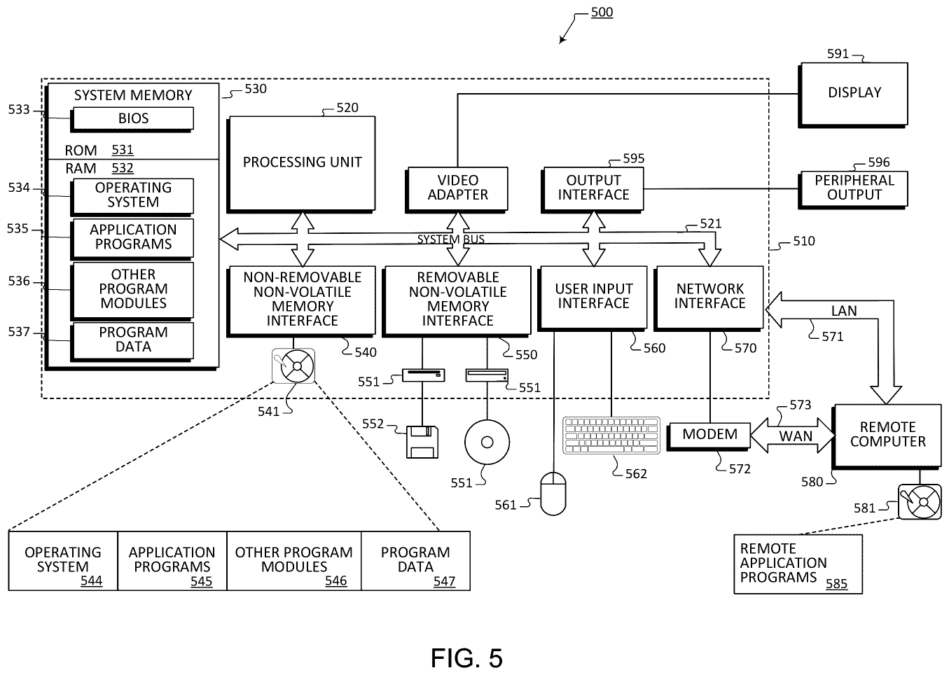

FIG. 5 depicts an example of a suitable computing system environment 500 on which aspects of the invention may be implemented. Such a computing environment may represent a home computer, a tablet, a mobile device, a server and/or any another computing device.

The computing system environment 500 is only one example of a suitable computing environment and is not intended to suggest any limitation as to the scope of use or functionality of the invention. Neither should the computing environment 500 be interpreted as having any dependency or requirement relating to any one or combination of components illustrated in the illustrative operating environment 500.

Aspects of the invention are operational with numerous other general purpose or special purpose computing system environments or configurations. Examples of well-known computing systems, environments, and/or configurations that may be suitable for use with the invention include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

With reference to FIG. 5, an illustrative system 500 for implementing aspects of the invention includes a general purpose computing device in the form of a computer 510. Components of computer 510 may include, but are not limited to, a processing unit 520, a system memory 530, and a system bus 521 that couples various system components including the system memory to the processing unit 520. The system bus 521 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 510 typically includes a variety of computer readable media. Computer readable media can be any available media that can be accessed by computer 510 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 510. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 530 includes computer storage media in the form of volatile and/or nonvolatile memory such as read only memory (ROM) 531 and random access memory (RAM) 532. A basic input/output system 533 (BIOS), containing the basic routines that help to transfer information between elements within computer 510, such as during start-up, is typically stored in ROM 531. RAM 532 typically contains data and/or program modules that are immediately accessible to and/or presently being operated on by processing unit 520. By way of example, and not limitation, FIG. 5 illustrates operating system 534, application programs 535, other program modules 536, and program data 537.

The computer 510 may also include other removable/non-removable, volatile/nonvolatile computer storage media. By way of example only, FIG. 5 illustrates a hard disk drive 541 that reads from or writes to non-removable, nonvolatile magnetic media, a magnetic disk drive 551 that reads from or writes to a removable, nonvolatile magnetic disk 552, and an optical disk drive 555 that reads from or writes to a removable, nonvolatile optical disk 556 such as a CD ROM or other optical media. Other removable/non-removable, volatile/nonvolatile computer storage media that can be used in the illustrative operating environment include, but are not limited to, magnetic tape cassettes, flash memory cards, digital versatile disks, digital video tape, solid state RAM, solid state ROM, and the like. The hard disk drive 541 is typically connected to the system bus 521 through a non-removable memory interface such as interface 540, and magnetic disk drive 551 and optical disk drive 555 are typically connected to the system bus 521 by a removable memory interface, such as interface 550.

The drives and their associated computer storage media discussed above and illustrated in FIG. 5, provide storage of computer readable instructions, data structures, program modules and other data for the computer 510. In FIG. 5, for example, hard disk drive 541 is illustrated as storing operating system 544, application programs 545, other program modules 546, and program data 547. Note that these components can either be the same as or different from operating system 534, application programs 535, other program modules 536, and program data 537. Operating system 544, application programs 545, other program modules 546, and program data 547 are given different numbers here to illustrate that, at a minimum, they are different copies.

A user may enter commands and information into the computer 510 through input devices such as a keyboard 562 and pointing device 561, commonly referred to as a mouse, trackball or touch pad. Other input devices (not shown) may include a microphone, joystick, game pad, satellite dish, scanner, or the like. These and other input devices are often connected to the processing unit 520 through a user input interface 560 that is coupled to the system bus but may be connected by other interface and bus structures, such as a parallel port, game port or a universal serial bus (USB). A monitor 591 or other type of display device is also connected to the system bus 521 via an interface, such as a video interface 590. In addition to the monitor, computers may also include peripheral output devices 596 such as speakers and printers, which may be connected through an output peripheral interface 595.

The computer 510 may operate in a networked environment using logical connections to one or more remote computers, such as a remote computer 580. The remote computer 580 may be a personal computer, a server, a router, a network PC, a peer device or other common network node, and typically includes many or all of the elements described above relative to the computer 510, although only a memory storage device 581 has been illustrated in FIG. 5. The logical connections depicted in FIG. 5 include a local area network (LAN) 571 and a wide area network (WAN) 573 but may also include other networks. Such networking environments are commonplace in offices, enterprise-wide computer networks, intranets and the Internet.

When used in a LAN networking environment, the computer 510 is connected to the LAN 571 through a network interface or adapter 570. When used in a WAN networking environment, the computer 510 typically includes a modem 572 or other means for establishing communications over the WAN 573, such as the Internet. The modem 572, which may be internal or external, may be connected to the system bus 521 via the network interface 570, or other appropriate mechanism. In a networked environment, program modules depicted relative to the computer 510, or portions thereof, may be stored in the remote memory storage device. By way of example, and not limitation, FIG. 5 illustrates remote application programs 585 as residing on memory device 581. It will be appreciated that the network connections shown are illustrative and other means of establishing a communications link between the computers may be used.

The various methods or processes outlined herein may be implemented in any suitable hardware. Additionally, the various methods or processes outlined herein may be implemented in a combination of hardware and of software executable on one or more processors that employ any one of a variety of operating systems or platforms. For example, the various methods or processes may utilize software to instruct a processor to determine a spatial position (e.g., based on image matching, odometry, etc.), to extract features from one or more images, to communicate with a data repository, to perform speech recognition, to perform speech synthesis, to compress and/or decompress image and/or video data, or combinations thereof. Example of such approaches are described above. However, any suitable combination of hardware and software may be employed to realize any of the embodiments discussed herein.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

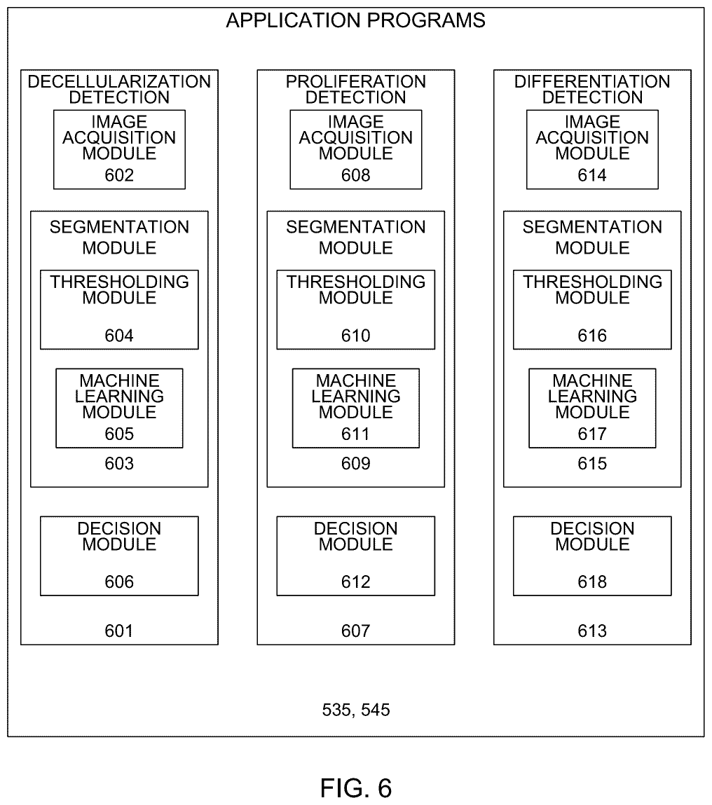

With reference to FIG. 6, a computer system 105 may be configured with application programs 535, 545 for decellularization detection 601, proliferation detection 607, and differentiation detection 613. The decellularization detection program 601 may comprise an image acquisition module 602 which receives digital data from the ultrasound probe 103a associated with the decellularization bioreactor 100a, and may conduct processing like filtering, noise reduction, and enhancement to improve image quality. The image acquisition module 602 may also apply any necessary calibration to correct device-specific distortions and convert the image to the desired format and resolution. Finally, the module 602 may be configured to apply advanced processing techniques like contrast adjustment, color correction, and feature extraction as well as add labels, markers, or annotations as needed for analysis or presentation.

The decellularization detection program 601 may further comprise a segmentation module 603 for partitioning an image into multiple segments or regions to isolate specific areas of interest within the image. The segmentation module may include a thresholding sub-module 604 for converting the acquired image to a binary image by setting one or more threshold values to separate pixel intensities into distinct regions to classify pixels based on their intensity values where pixel intensity is an indication the degree of decellularization. The module 603 may also include routines for conducting edge detection.

The segmentation module 603 preferably includes a trained machine learning sub-module 605 to classify and label segments. The sub-module 605 is trained to segment images using supervised learning techniques, where the model learns from labeled datasets that contain both the input images and their corresponding segmentation masks (ground truth) from large and diverse data set of images. For each image, a corresponding segmentation mask is created, often by human annotators. The mask indicates the segments of interest within the image. Techniques such as rotation, scaling, flipping, and cropping may be applied to the training images and masks to increase the diversity of the dataset and improve the robustness of the model.

The machine learning model is preferably a Convolutional Neural Network (CNN) due to the CNN's ability to capture spatial hierarchies in images. However, in some embodiments a fully convolutional network (FCN) may be used. These are adapted CNNs where the fully connected layers are replaced by convolutional layers, enabling the model to produce a segmentation map. Another possible model is U-Net which consists of an encoder-decoder structure with skip connections to preserve spatial information. The model may be trained using gradient descent and backpropagation. During each iteration, the model predicts the segmentation mask for a batch of input images, and a loss function quantifies the difference between the predicted masks and the ground truth. The gradients of the loss function with respect to the model parameters are computed and used to update the parameters in a direction that reduces the loss. The trained model may then be evaluated on a separate validation set to monitor its performance and tune hyperparameters. After training, the model is tested on a holdout test set to assess its generalization ability.

The decellularization detection program 601 may preferably include a decision module 606 which is configured to determine whether tissue within the decellularization reaction chamber 102a is sufficiently decellularized. This determination is based upon a pre-determined criteria.

The proliferation detection program 607 and the differentiation detection program 613 have similar modules and sub-modules. The proliferation detection programs 607 also includes and image acquisition module 608 for acquiring image data, conducting image processing as described above with respect to the decellularization detection program. The proliferation detection program also includes a segmentation module 609 with a thresholding module 610 where, in this case, pixel intensity is indicative of the presence of tissue cells versus a scaffold, thus, indicating a degree of cellular confluence. A machine learning module 611 for classification and labeling of scaffolding versus tissue cells. This module 611 is trained in a manner similar that described above, except using cell-culture imagery. The proliferation detection program 607 also preferably includes a decision module 612 configured to determine whether tissue within the proliferation reaction chamber 102b has achieved cellular confluence to a pre-determined degree.

The differentiation detection program 613 also comprises an image acquisition module 614, a segmentation module 615, and a decision module 618. The segmentation module 615 preferably comprises a thresholding module 616 and a machine learning module 617. In the thresholding module 616, pixel intensity is indicative of a type of tissue, i.e., muscle tissue (myocytes), or fat tissue (lipocytes). The machine learning module 617 has been trained to classify and label imagery regions based upon pixel intensity as to whether a region indicates the type of tissue. The differentiation detection program 613 also preferably includes a decision module 618 configured to determine whether tissue within the differentiation reaction chamber 102c has achieved differentiation to a pre-determined degree.

As described above and shown in the associated drawings, the present invention comprises a bioreactor system for production of cell-cultured meat and related methods. While particular embodiments have been described, it will be understood, however, that any invention appertaining to the apparatus, system, and/or methods described is not limited thereto, since modifications may be made by those skilled in the art, particularly in light of the foregoing teachings. It is, therefore, contemplated by the appended claims to cover any such modifications that incorporate those features or those improvements that embody the spirit and scope of the invention.

What is claimed is:

1. A bioreactor comprising:
a chamber for containing a reaction, the reaction being one of a decellularization reaction, a proliferation reaction, and a differentiation reaction; and
an ultrasound probe configured to emit ultrasonic pulses into the chamber to detect one of a degree of decellularization, a degree of proliferation, and a degree of differentiation.

2. The bioreactor of claim 1, wherein the ultrasound probe emits pulses having a frequency of up to about 100 MHz.

3. The bioreactor of claim 1, wherein the ultrasound probe is in a connection with a processor with memory and software operative to provide one or more digital images indicative of the degree of decellularization, the degree of proliferation, or the degree of differentiation.

4. The bioreactor of claim 3, wherein the software is operative to analyze the one or more digital images to detect at least one of the degree of decellularization, the degree of proliferation, or the degree of differentiation.

5. The bioreactor of claim 4, wherein the software comprises a trained machine learning software module.

6. A method of manufacturing cell-cultured meat comprising the steps of:
decellularizing tissue;
detecting a degree of decellularization of the tissue by propagating ultrasonic pulses into the tissue;
seeding primer cells onto a scaffold;
detecting a degree of cellular confluence by propagating ultrasonic pulses into the seeded scaffold;
inducing differentiation of cells populating confluent scaffolds; and
detecting a degree of differentiation by propagating ultrasonic pulses into confluent scaffolds.

7. The method of claim 6, wherein each step of detecting further comprises the steps of:
acquiring two-dimensional ultrasound image data;
defining a threshold based upon intensity; and
segmenting a portion of the data based upon the threshold.

8. The method of claim 7, wherein the step of segmenting is performed by a trained machine learning software module.

9. The method of claim 6, wherein the tissue is from a plant.

10. The method of claim 9, wherein the plant is one of spinach, broccoli, and aloe.

11. The method of claim 6 executed as a real time and continuous method capable of providing a continuous production line for cell-cultured meat with a real time quality control monitoring.

12. A system for manufacturing cell-cultured meat comprising:
a decellularization bioreactor for containing decellularizing tissue;
an ultrasound probe configured to emit ultrasound pulses into the decellularizing tissue to detect a degree of decellularization thereof;
a proliferation bioreactor for containing a scaffold seeded with primer cells;
an ultrasound probe configured to emit ultrasound pulses into the scaffold to detect a degree of confluence thereof;
a differentiation bioreactor for containing a process for differentiating cells populating a confluent scaffold; and
an ultrasound probe configured to emit ultrasound pulses into the differentiating cells to detect a degree of differentiation thereof.

13. The system of claim 12, wherein the ultrasound probe emits pulses having a frequency of up to about 100 MHz.

14. The system of claim 12, wherein the tissue is a plant tissue.

15. The system of claim 14, wherein the plant tissue is one of spinach, broccoli, and aloe.

16. The system of claim 12, further comprising a computer-based system having a machine-readable memory coupled to a processor, the memory having program instructions stored thereon that, upon execution cause the computer system to:
receive two-dimensional ultrasound image data;
define a threshold based upon intensity; and
segment a portion of the data based upon the threshold.

17. The system of claim 16, wherein the instruction to segment a portion of the data is performed by a trained machine learning software module.
